(19) United States
(12) Patent Application Publication
(10) Pub. No.: US 2013/0158573 A1
(43) Pub. Date: Jun. 20, 2013

(54) OPHTHALMIC SURGERY INSTRUMENT

(71) Applicants: Elias ZAIDMAN, Mexico City (MX); David FRILEUX, Autry-Issards (FR)

(72) Inventors: Elias ZAIDMAN, Mexico City (MX); David FRILEUX, Autry-Issards (FR)

(73) Assignee: MORIA SA, Antony (FR)

(21) Appl. No.: 13/336,649

(22) Filed: Dec. 23, 2011

(30) Foreign Application Priority Data
Dec. 16, 2011 (FR) 11/61810

(51) Int. Cl. A61F 9/007

(52) U.S. Cl. 606/166

(57) ABSTRACT

The invention relates to an ophthalmic surgery instrument (1; 101) for making a substantially circular cut in an internal part (110) of an eye (109), in particular for capsulorhexis, comprising a handle (2; 102) at the end of which there extends a rod (3; 103) carrying a blade (4; 104) designed to be introduced into the eye (109) via an incision (107) that has been formed in the latter, characterized in that the blade (4; 104) has a loop shape with dimensions substantially equal to those of the cut, and the instrument comprises means for passing the blade through the incision.

### TECHNICAL FIELD

Capsulorhexis is generally carried out in cataract surgery and involves making a circular cut in the capsule enclosing the crystalline lens, such that the latter can be extracted therefrom before being replaced by an artificial intraocular implant.

The capsulorhexis procedure, the extraction of the crystalline lens and the fitting of the intraocular implant in the capsule are performed via an incision made in the cornea.

The incision being quite small, the crystalline lens is divided into fragments that are aspirated through the cut and the incision, while the intraocular implant is generally in the form of a lens that is deformed in order to permit its introduction into the capsule via the incision before returning to its normal shape once it has been placed in the capsule.

The capsule is usually cut open by means of an instrument composed of a needle with a curved end, or cystotome, introduced into the incision made in the cornea. It is also known to use forceps whose free ends are curved.

The practical experience of ophthalmic surgeons has shown that the regularity of the cut is a factor that limits the risk of complications: the cut must be centred, have a clean circular edge without tear-out, and have a diameter adapted to the eye being operated on and to the implant being fitted.

A number of solutions have been put forward for obtaining a regular cut.

Thus, for surgeons working with a microscope, it is known to display, in the viewing device of the microscope, a circular contour centred on the eye of the patient in such a way that the surgeon visualizes the position and dimensions of the cut that is to be made. Although displaying the contour of the cut can help the surgeon, the regularity of the cut still depends to a large extent on the surgeon's ability in handling the cutting tool.

The use of forceps has, moreover, the disadvantage of causing leaks in the region of the incision, necessitating the simultaneous introduction of liquid in order to maintain the intraocular pressure.

### OBJECT OF THE INVENTION

An aim of the invention is to make available a means by which it is easier to make a regular surgical cut.

### BRIEF DESCRIPTION OF THE INVENTION

To this end, the invention relates to an ophthalmic surgery instrument for making a substantially circular cut in an internal part of an eye, in particular for capsulorhexis, comprising a handle at the end of which there extends a rod carrying a blade designed to be introduced into the eye via an incision that has been formed beforehand in the latter. The blade has a loop shape with dimensions substantially equal to those of the cut, and the instrument comprises means for passing the blade through the incision in successive linear sections.

Such an instrument allows a regular cut to be made, overcoming the lack of ability of the surgeon. By virtue of the means of passage, the blade is introduced into the incision in successive linear sections, which makes it possible to maintain an incision of small dimensions. The risks of post-operative infection, which are closely linked to the size of this incision, are therefore reduced.

Other features and advantages of the invention will become clear on reading the following description of two particular and non-limiting illustrative embodiments of the invention.

### BRIEF DESCRIPTION OF THE DRAWINGS

Reference is made to the attached drawings, in which:

FIG. 1 is a side view of a surgical instrument comprising a retracted blade, according to a first embodiment of the invention;

FIG. 2 is a side view of the instrument according to FIG. 1, with the blade deployed;

FIG. 3 is a schematic side view of the ophthalmic surgery instrument according to a second embodiment of the invention;

FIG. 4 is a schematic front view of the instrument according to FIG. 3;

FIG. 5 is a schematic sectional and perspective view of an eye in which the instrument according to FIGS. 3 and 4 is placed;

FIG. 6 is a schematic view of the eye after use of the instrument.

### DETAILED DESCRIPTION OF THE INVENTION

The invention is described here on the basis of an ophthalmic surgery instrument for capsulorhexis.

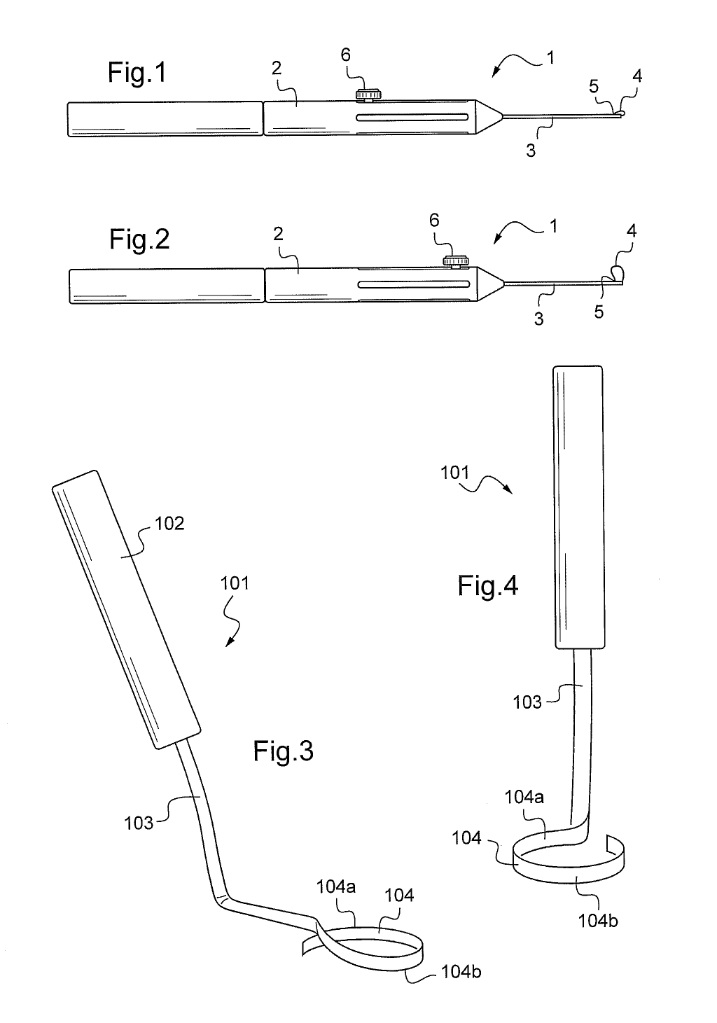

Referring to FIGS. 1 and 2, the instrument comprises a handle 2 forming a gripping zone, from which there extends a rod 3 to which a blade 4 is connected.

The blade 4 is deformable between a state of use, in which the blade 4 has a loop shape, and a state of non-use, in which the blade 4 is at least partially straightened out so as to extend rectilinearly. The blade 4 comprises a sharp lower edge along its entire length.

The rod 3 is hollow in such a way as to receive and permit sliding of the blade 4, of which a first end is free and a second end is connected to a control slide 6 sliding in the handle 2. The blade 4 is thus mounted on the rod 3 so as to be movable between an insertion position, in which the blade in its state of non-use is retracted inside the rod 3, and a cutting position, in which the blade 4 in its state of use extends out of the rod 3.

In a position of use of the instrument 1, as is illustrated in FIG. 2, the blade 4 is deployed by the movement of the control slide 6. By virtue of the material from which the blade 4 is made, for example a shape-memory material or a superelastic material, the blade 4 changes from its state of non-use to its state of use spontaneously as it leaves the rod 3. To make it easier to form the loop shape, it is possible for the end of the blade 4 opposite the control slide 6 to be fixed to the rod 3 at a point 5 near the free end of the rod 3.

During the operation, the surgeon makes an incision in the cornea and introduces the rod 3 of the instrument into the latter while the blade 4 is in the insertion position.

The control slide is then pushed in order to cause the blade 4 to move out. It will be appreciated that in this way the blade 4 passes through the incision in successive linear sections as it leaves the rod 3, said rod 3 protecting the incision from the sharp edge of the blade 4 by avoiding any contact between these.

As it leaves the rod 3, the blade 4 returns to its state of use. Once the blade 4 is again in the shape of a loop, the surgeon simply has to place it against the capsule and apply a slight rotation movement in order to completely cut open the capsule.

The surgeon then manoeuvres the control slide 6 in order to retract the blade 4 into the rod 3 and then withdraws the rod 3 from the incision.

The operation can then continue.

FIGS. 3 and 4 illustrate a second embodiment of the instrument, where the reference number for elements similar to the first embodiment are increased by a hundred.

In this embodiment, the instrument 101 comprises a handle 102 at the end of which there extends a solid rod 103. The rod 103 carries a blade 104 of helical shape, of which the helix angle is sufficiently great to obtain an open loop.

The blade 104 is made of metal and comprises an upper edge 104a and a lower edge 104b, of which only the lower edge 104b is sharp along its entire length.

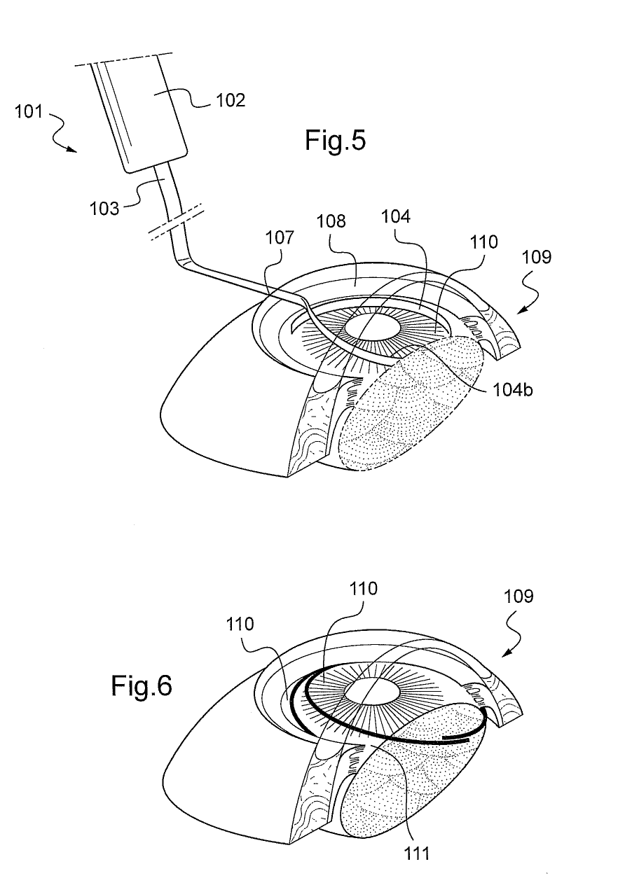

FIGS. 5 and 6 are schematic illustrations of two different surgical steps of a cataract procedure in which the instrument 101 according to the second embodiment is used.

These steps have been preceded by formation of the corneal incision 107, for example with the aid of a scalpel, for accessing the anterior chamber 108 of the eye 109.

The surgeon then begins by inserting the free end of the blade 104 through the incision 107 and then applies a helical movement to the handle 102 (in a screwing direction) in order to pass the blade 104 in successive linear sections into the incision 107, in such a way as to introduce the whole blade 104 into the anterior chamber 108. The blade 104 is then placed on the anterior capsule 110 of the crystalline lens and centred thereon, as is illustrated in FIG. 5. The blade 104 preferably has sufficient flexibility to allow it to be “flattened” against the capsule during this manoeuvre, in such a way that its sharp edge is in contact with the capsule along its entire length.

The cut is made as above, and the blade is withdrawn from the eye by a reverse helical movement (i.e. in an unscrewing direction).

The use of the instrument 101 according to this second embodiment may pose a risk of damaging the incision 107 through which the blade 104 passes, on account of the presence of the sharp edge 104b. Thus, it is conceivable to add a guide (not shown) in the area of the incision 107, making it possible to avoid any needless and/or inadvertent cutting of the cornea during the introduction of the blade 104.

It will be noted that, once the entire length of the sharp edge 104b of the blade 104 is positioned on the capsule 110, the surgeon applies gentle rotation movements, in one direction and then in the other, in such a way as to cut open the capsule 110 (curvilinear capsulorhexis). These movements can be obtained by setting the blade 4 in vibration, preferably by means of an ultrasonic or piezoelectric vibratory device acting on the handle 2 or on the rod 3.

Referring to FIG. 6, the cut-open capsule 110 can be withdrawn and the core of the crystalline lens 111 becomes accessible, such that the surgeon is able to continue the operation.

The deployment and the retraction of the deformed blade 4 in the rod 3 of the first embodiment and the open-loop shape of the blade 104 of the second embodiment constitute means of passing the blade through the incision in successive linear sections, which permit an incision 107 with a small size.

Of course, the invention is not limited to the embodiments described above and can be varied in ways that will be evident to a person skilled in the art without departing from the scope of the invention as defined by the claims.

Although the helical blade 104 of the second embodiment is made of metal, it can also be made of ceramic.

The vibratory device, which can be used in both of the embodiments described, can be accommodated in the handle or can be external to the latter.

Finally, to make it easier to cut open the capsule, it is also conceivable for the blade to comprise a heating part.

1. Ophthalmic surgery instrument (1; 101) for making a substantially circular cut in an internal part (110) of an eye (109), in particular for capsulorhexis, comprising a handle (2; 102) at the end of which there extends a rod (3; 103) carrying a blade (4; 104) designed to be introduced into the eye (109) via an incision (107) that has been formed beforehand in the latter, characterized in that the blade (4; 104) has a loop shape with dimensions substantially equal to those of the cut, and the instrument comprises means for passing the blade through the incision in successive linear sections.

2. Instrument (1; 101) according to claim 1, in which the blade (104) extends helically in order to form an open loop allowing the blade to pass through the incision in a helical movement.

3. Instrument (101) according to claim 2, in which the blade (104) is made of ceramic.

4. Instrument (101) according to claim 2, in which the blade (104) is made of metal.

5. Instrument (1) according to claim 1, in which the blade (4) is deformable between a state of use, in which the blade has the loop shape, and a state of non-use, in which the blade is at least partially straightened out, and the blade is mounted on the rod (3) in order to be movable between an insertion position, in which the blade in its state of non-use is at least partially retracted inside the rod, and a cutting position, in which the blade in its state of use extends out of the rod.

6. Instrument (1) according to claim 5, in which the deployment and the retraction of the loop are activated by a control (6) arranged in the handle (2).

7. Instrument (1) according to claim 6, in which the blade (4) is made of a shape-memory material.

8. Instrument (1) according to claim 6, in which the blade (4) is made of a superelastic material.

9. Instrument (1; 101) according to claim 1, in which the blade (4; 104) comprises a sharp lower edge (104b) along its entire length.

10. Instrument (1; 101) according to claim 1, in which the blade (4; 104) is set in vibration by a vibratory device connected to the handle (2; 102).

11. Instrument (1; 101) according to claim 1, in which the blade (4; 104) comprises a heating part.
